(19) United States
(12) Patent Application Publication
(10) Pub. No.: US 2025/0387240 A1
(43) Pub. Date: Dec. 25, 2025

(54) Method and system of monitoring a medical prosthesis

(71) Applicant: Lipalo Mokete, Bryanston (ZA)

(72) Inventor: Lipalo Mokete, Bryanston (ZA)

(21) Appl. No.: 19/315,190

(22) Filed: Aug. 29, 2025

Related U.S. Application Data
(63) Continuation-in-part of application No. 19/263,201, filed on Jul. 8, 2025.
Application No. 19/263,201 is a continuation of application No. 17/408,198, filed on Aug. 20, 2021, now Pat. No. 12,370,065.
Application No. 17/408,198 is a continuation-in-part of application No. PCT/IB2020/050729, filed on Jan. 30, 2020.

(30) Foreign Application Priority Data
Feb. 22, 2019 (ZA) 2018/05590
Aug. 29, 2024 (ZA) 2024/06670

(51) Int. Cl. A61F 2/46; A61F 2/30; A61F 2/38

(52) U.S. Cl. CPC A61F 2/468; A61F 2/30771; A61F 2/3859; A61F 2/389

(57) ABSTRACT

The invention relates to a medical prosthesis monitoring system 10 and method 60 of synchronizing, labelling, and predicting activity based on data generated by dual Inertial Measurement Unit (IMU) sensors 13 implanted in a knee replacement prosthesis 11. The present invention provides accurate tracking of knee replacement activity and near-real-time prediction of specific activity thereby providing objective data on the quantity and frequency of such activities. Specifically, the system 10 temporally synchronises knee activity related accelerometer and gyroscope data generated by the two or more sensors 13 placed each in the femoral and tibial components of the knee replacement prosthesis 11. This data is used to train a multimodal deep neural network architecture which combines a Long-Short Term Memory (LSTM) network on the filtered timeseries data with a Convolutional Neural Network (CNN) on spectrograms of the timeseries data to accurately predict activity and biomechanics of the knee replacement.

### CROSS-REFERENCE TO RELATED APPLICATIONS

This application, which claims priority to ZA Application No. 2024/06670, filed 29 Aug. 2024, is a continuation-in-part of application Ser. No. 19/263,201 filed on 8 Jul. 2025 which is a continuation of application Ser. No. 17/408,198, filed on 20 Aug. 2021, which claims priority to PCT Application No. PCT/IB2020/050729 filed on 30 Jan. 2020 and ZA Application No. 2018/05590, filed on 22 Feb. 2019. The entire content of these applications is incorporated herein by reference in their entireties.

### FIELD OF INVENTION

The invention relates generally to medical prosthetics and more specifically to a method and system for monitoring a condition and activity of an endoprosthesis fitted with at least one electronic device or sensor.

### BACKGROUND OF INVENTION

Contemporary knee replacements in their basic form consist of a femoral component, a tibia component, and a polyethylene liner. Knee replacements have been implanted into patients with end stage degenerative conditions of the knee to treat arthritic conditions for the past 50 years. The aim of knee replacement surgery is to reduce pain and disability in the recipient of the prosthesis and ultimately, to restore quality of life and return the recipient to the pre-disease state. This ideal is seldomly achieved.

Current validated methods for assessing activity of the knee following replacement surgery rely on subjective patient reported outcome measures.

This standard has limitations including psychosocial influences, ceiling, and floor effects. Widespread availability of Inertial Measurement Units (IMUs) as activity trackers has seen their increasing adoption as instruments to measure activity in an objective manner following knee replacement surgery. The IMU's typically provide information on quantity and intensity of activity, gait metrics, joint angles, and postural stability. The objective measures of activity can be used to complement the subjective assessment for more comprehensive activity evaluation. Currently, the quantity of activity is generally expressed in broad terms without specifying the ambulatory Activity of Daily Living (ADL) and intensity is stated as either vigorous, moderate or sedentary. When mention is made of specific ADL, these are mostly confined to gait metrics (step frequency, gait length, gait speed, gait cycle).

The concept of specific knee activity profiles focuses on quantifying the frequency of knee activities that form part of activities of daily living (ADL) undertaken over a defined period. Patients with knee replacements spend 8% to 10% of their time during the day pursuing dynamic activities. These ADL are not limited to walking and examples include standing, sitting, stair climbing, stair descent, running, non-loaded flexion and extension of the knee joint, and transitioning between activities. Prior knowledge of knee activity profiles during health forms the most accurate objective measure to benchmark activity should knee activity subsequently become impaired due to injury or arthritis. Activity profiles can be the cornerstone of a personalised rehabilitation program following knee replacement surgery.

Outside of knee replacements, activity profiles are important in relaying objective information about performance of specified activities that are part of a rehabilitation regime following knee injury and avoidance of activities that would exacerbate the injury. Furthermore, customised activity profiles enhance motivation and engagement.

WO 2023/278775 describes an intelligent implant in the form of a knee arthroplasty device for patients undergoing knee replacement which includes an inertial measurement unit (IMU). The IMU captures orientation and movement information of the device (and the knee in which it is implanted) and uploads this data periodically to a central location where it can be processed and analysed. Based on signal processing performed on the data, the systems and methods detect walking activity, partition walking activity into steps, extract clinically relevant features of a step, and how those step features can be used to evaluate patient prognosis including pain, mobility, and stiffness. The above-described implant has the drawback that the IMU is located medially in the knee prosthesis which makes gaining access to it difficult to impossible. Therefore, if the device malfunctions or needs to be serviced or replaced, a complete revision of the knee replacement is necessitated which is obviously undesirable. Furthermore, only a single IMU is used in the implant. In addition, activity trackers such as these typically rely on detecting peaks and valleys in graphs generated from IMU data. Threshold values are then set to detect, for example, the number of steps taken or stairs climbed. The problem with this approach is the need to constantly vary the thresholds to maintain accuracy of reporting.

The present invention aims, at least to some extent, to alleviate the drawbacks discussed above.

### SUMMARY OF INVENTION

In accordance with a first aspect of the invention, there is provided a computer-implemented method of monitoring a medical prosthesis which includes at least two implantable electronic devices implanted in the medical prothesis, the method including:
obtaining data from the implantable electronic devices;
performing, using a computing device, temporal-spatial analysis of the obtained data;
performing activity recognition using machine learning techniques; and
comparing recognised activities against trained machine learning models in order to identify anomalies associated with the medical prosthesis.

The method may include the prior step of synchronizing time-stamped data obtained from both of the respective implantable electronic devices.

The step of performing temporal-spatial analysis of the obtained data may include transforming, using the computing device, the obtained data into spectrograms.

The method may further include processing, using the computing device, the spectrograms through a convolutional neural network in order to identify specific temporal-spatial patterns.

The step of performing temporal-spatial analysis may include:
performing temporal analysis of the obtained data using a machine learning model in a temporal branch; and,
in parallel to that, performing temporal-spatial analysis of the obtained data using spectrograms in a temporal-spatial branch.

Performing temporal analysis in the temporal branch may include the prior step of filtering the obtained data. Also, performing temporal-spatial analysis in the temporal-spatial branch may include processing the spectrograms through a trained convolutional neural network in order to identify specific temporal-spatial patterns.

The method may include late-fusing the respective branches by concatenating processed data from the temporal branch and the temporal-spatial branch to integrate features learned by each branch in order to accurately recognize activity patterns without needing manually to adjust thresholds.

Performing activity recognition may include creating a probability distribution for all activity classes, using a multi-class classification algorithm, such that a highest probability in the distribution becomes the activity prediction.

The step of comparing recognised activities against trained machine learning models may include using a K-means clustering machine learning algorithm to partition datasets.

The method may further include using t-Distributed Stochastic Neighbor Embedding (t-SNE) plotting visually to identify anomalies associated with the medical prosthesis.

Also, the method may include performing cluster-distance anomaly detection by plotting distances to cluster centres for recognised activities.

The medical prosthesis may be a knee prosthesis. It will be appreciated that the same method described above may be applied to other replacement body parts or prostheses.

The method may further include the prior step of training the machine learning models using data obtained from patients with well-functioning medical protheses.

In accordance with another aspect of the invention, there is provided a medical prosthesis monitoring system which includes:
at least two implantable electronic devices mounted to a medical prothesis fitted to, or mountable to a subject; and
a remote computing device which is communicatively coupled to a wireless communication module of the implantable electronic devices and is configured to:
   wirelessly interrogate the implantable electronic devices to obtain data measured by electromechanical motion sensors of the implantable electronic devices;
   perform temporal-spatial analysis of the obtained data;
   perform activity recognition using machine learning techniques; and
   compare recognised activities against trained machine learning models in order to identify anomalies associated with the medical prosthesis.

At least one of the implantable electronic devices may be implanted into an augment attached to part of the medical prosthesis.

The remote computing device may be configured to transform, using a processor, the obtained data into spectrograms.

The remote computing device may be further configured to:
perform, using the processor, temporal analysis of the obtained data using a machine learning model in a temporal branch; and,
in parallel to that, perform, using the processor, temporal-spatial analysis of the obtained data using spectrograms in a temporal-spatial branch.

Furthermore, the remote computing device may be configured to perform cluster-distance anomaly detection by plotting distances to cluster centres for recognised activities.

Finally, the invention extends to a non-transitory computer-readable storage medium having program instructions stored thereon, which, when executed by a computing device, enable the computing device to perform the method steps described above.

### BRIEF DESCRIPTION OF DRAWINGS

The invention will now be further described, by way of example, with reference to the accompanying drawings.

In the drawings:

FIG. 1 shows a functional block diagram of a medical prosthesis monitoring system in accordance with an aspect of the invention;

FIG. 2 shows a three-dimensional view of a prior art configuration of a knee prosthesis comprising a femoral component and a tibial component;

FIG. 3 shows a three-dimensional, partially exploded view of an endoprosthesis;

FIGS. 4A and 4B show side elevations of an endoprosthesis fitted to a patient;

FIG. 5 shows a three-dimensional view of an alternative embodiment of an endoprosthesis including augments;

FIG. 6 shows a flow diagram of a method of monitoring a medical prosthesis in accordance with the invention;

FIG. 7 illustrates time synchronization of data obtained from two separate IMUs;

FIG. 8 illustrates plots of filtered and unfiltered data obtained from 12 channels of the respective IMUs;

FIG. 9 illustrates spectrograms computed from unfiltered data;

FIG. 10 shows a block diagram of machine learning model architecture of the medical prosthesis monitoring system;

FIG. 11 shows a plot illustrating clustering of activities to identify patterns and anomalies; and

FIG. 12 illustrates cluster-distance plots for various activities.

### DETAILED DESCRIPTION OF AN EXAMPLE EMBODIMENT

The following description of the invention is provided as an enabling teaching of the invention. Those skilled in the relevant art will recognise that many changes can be made to the embodiments described, while still attaining the beneficial results of the present invention. It will also be apparent that some of the desired benefits of the present invention can be attained by selecting some of the features of the present invention without utilising other features. Accordingly, those skilled in the art will recognise that modifications and adaptations to the present invention are possible and can even be desirable in certain circumstances, and are a part of the present invention. Thus, the following description is provided as illustrative of the principles of the present invention and not a limitation thereof.

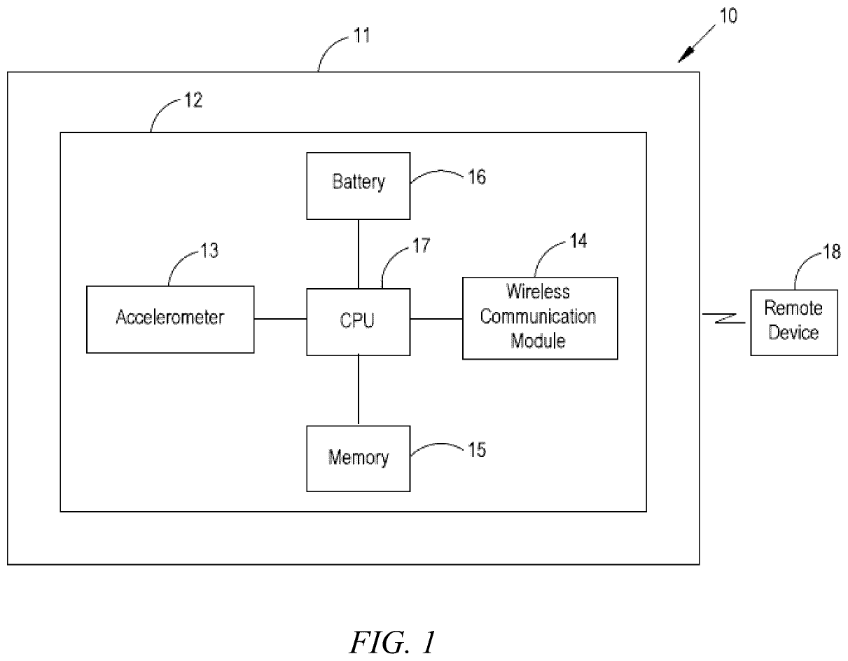

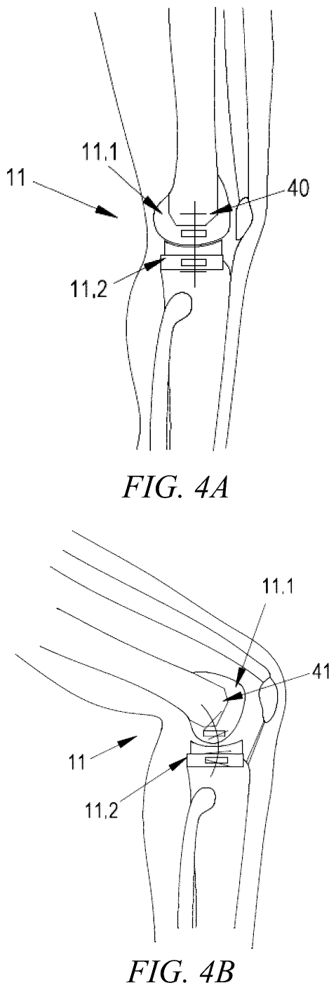

In FIG. 1 reference numeral 10 refers generally to a medical prosthesis monitoring system in accordance with the invention. The system 10 includes an endoprosthesis 11, which in this example embodiment is in the form of a knee prosthesis configured to be fitted as a replacement body part to a subject or patient (see FIGS. 4A and 4B). The knee prosthesis illustrated in this example happens to be a complete knee prosthesis. It is envisaged that the invention may also find application in partial knee prosthesis as well as in other replacement body parts. In this example embodiment, the medical prosthesis monitoring system 10 includes at least two implantable electronic devices 12.1, 12.2 (although only one implantable electronic device 12 has been illustrated in FIG. 1) which are operatively mounted to or received within separate cavities 35.1, 35.2 provided in the endoprosthesis 11, as will be explained in more detail below. The system 10 also includes an external remote computing device 18 which is configured wirelessly to interrogate the implantable electronic devices 12 in order to glean recordings or measurements from them. To this end, each implantable electronic device 12 includes a processor or CPU 17 and a wireless communication module 14 which is communicatively linked to the processor 17 and is configured to communicate with the remote computing device 18 using any suitable wireless communication protocol. The wireless communication module 14 may be laterally disposed within the cavity to ensure least possible interference between the wireless communication module 14 and the remote computing device 18. Each implantable electronic device 12 also includes at least one accelerometer or Inertial Measurement Unit (IMU) 13 which is configured to measure acceleration and rotation of at least part of the endoprosthesis 11. The accelerometer 13 may be a three-axis piezoelectric MEMS accelerometer. The implantable electronic device 12 also includes memory 15 for storing recorded measurements, data or readings of the accelerometer 13 and a power source in the form of a battery 16. The battery 16 may be a rechargeable battery. The battery 16 may be recharged, wirelessly through use of an inductive charger. Accordingly, a receiving circuit (not shown) may be connected to the rechargeable battery for coupling with an external inductive charger which is operatively brought into close proximity to the receiving circuit. Alternatively, power sources such as kinetic energy harvesters (not shown) may also be incorporated into the implantable electronic device 12 in order to recharge the battery 16. The IMU 13 is configured to record and/or measure vibration, shock, tilt and rotation amongst others.

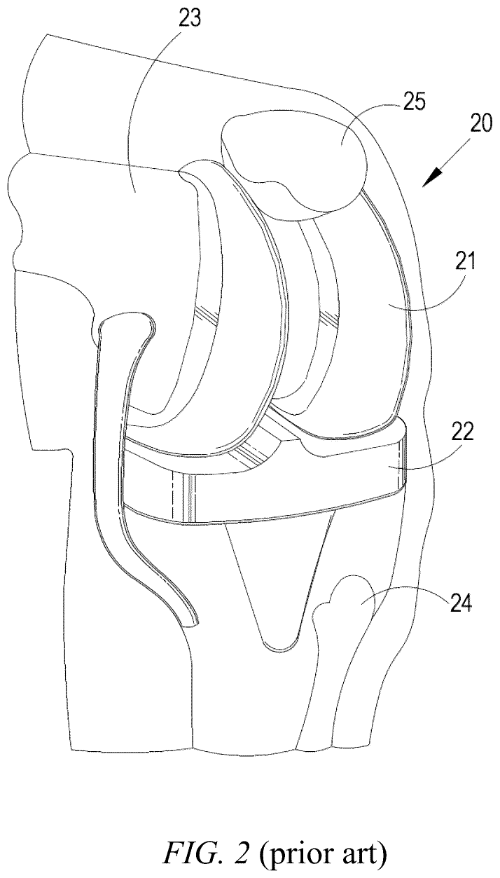

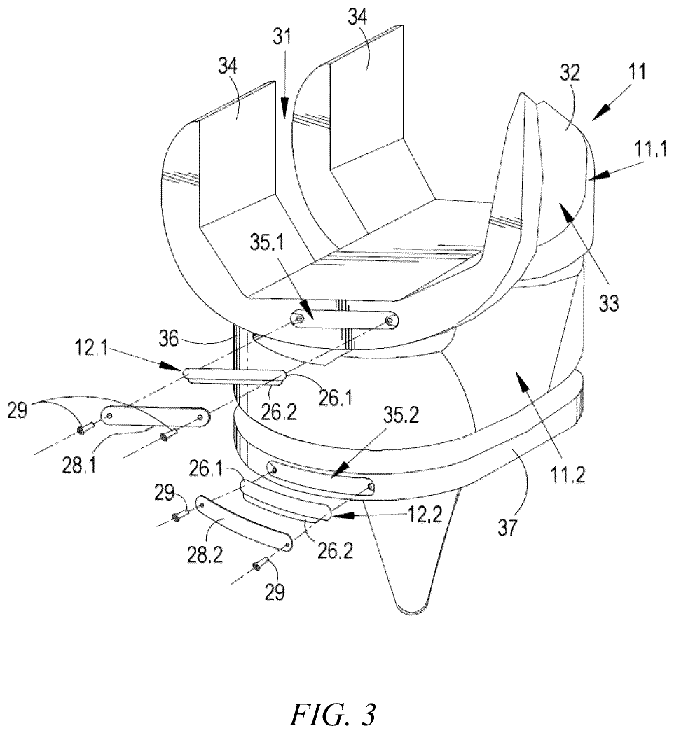

With reference to FIG. 2, a conventional, prior art knee prosthesis 20 includes a femoral component 21 which is attached to a degraded distal end of the patient's femur 23 and a tibial component 22 which is connected to a tibia 24 of the patient. The femoral component 21 articulates with the tibial component 22 in order to form an artificial or replacement knee joint. In the event of a total knee replacement, a patellar prosthesis 25 may also be provided. With reference to FIG. 3, the endoprosthesis 11 includes a first part in the form of a femoral component 11.1 and a second part in the form of a tibial component 11.2. The femoral component 11.1 and the tibial component 11.2 articulate to form a knee joint. As illustrated in FIG. 2, the endoprosthesis 11 may also include a patellar prosthesis fitted to a patella, although this has not been illustrated in FIG. 3. The femoral component 11.1 includes a convexly curved, C-shaped head 31. The head 31 has an anterior, pointed protrusion 32 which defines a prominent groove 33 for accommodating and facilitating tracking of the patella. The anterior, pointed protrusion 32 is joined to a pair of posteriorly bifurcating, convexly curved femoral condyles 34. As can be seen in FIG. 3, a laterally inwardly extending blind hole or cavity 35.1, having an oblong cross-section, is provided in a lateral aspect of one femoral condyle 34 of the femoral component 11.1 of the endoprosthesis 11. A first of the two implantable electronic devices 12.1 is operatively removably received or accommodated in the blind cavity 35.1. The blind cavity 35.1 has a length of 15 mm, a breadth of 3.5 mm and a depth of 15 mm. The tibial component 11.2 includes a tibial lining 36 which interfaces with the head 31 of the femoral component 11.1 and a tibial plate 37 which is secured to the tibia 24. A second, laterally inwardly extending blind hole or cavity 35.2 is provided in a lateral aspect of the tibial plate 37 of the tibial component 11.2. This second blind hole 35.2 is configured removably to accommodate the second implantable electronic device 12.2. The second blind hole 35.2 has a length of 15 mm, a breadth of 3.5 mm and a depth of 15 mm, i.e. similar dimensions to the first cavity 35.1. The medical prosthesis monitoring system 10 is therefore configured to measure, record and transmit relative acceleration data of the two implantable electronic devices 12.1, 12.2 and, hence, of the femoral component 11.1 and the tibial component 11.2.

Although it has not been illustrated in the Figures, it will be appreciated that additional accelerometers or implantable electronic devices 12 may be provided in or on the patellar prosthesis. Also, multiple accelerometers may be provided on either of, or both of the femoral and tibial components in dedicated cavities or openings. Each implantable electronic device 12 may include multiple accelerometers 13.

The blind cavities 35.1, 35.2 are provided in lateral aspects of the femoral component 11.1 and tibial component 11.2, respectively, due to the fact that it is an area of the knee that has the least soft tissue cover and is easily accessible. This renders the implantable electronic devices 12.1, 12.2 easily removable, and hence serviceable, for example to replace depleted batteries or malfunctioning components, by way of only minor surgery along any one of the surgical incision lines 40, 41 shown in FIGS. 4A and 4B respectively. In this way, a life span of the implantable electronic devices 12 can be extended without having to remove or interfere with, and potentially compromise, a well-functioning stable knee prosthesis. Furthermore, the accelerometers 13 installed in the endoprosthesis 11 are connected via a wireless digital communication interface to the remote device 18 and are configured to send or transmit accurate information about the type and intensity of activity of the knee to the remote computing device 18 via a thinnest aspect of the knee across the soft tissue using wireless transmission technology. Furthermore, there would be less tissue signal interference when harvesting data from the implantable electronic device 12 from this location. FIG. 4B illustrates how in an alternative embodiment of the endoprosthesis where the blind cavity is provided in the lateral aspect of the postero-lateral condyle, the blind cavity can still be easily accessed with the knee placed in slight flexion.

The endoprosthesis 11 is made of strong materials that fit the purpose of the implant. The blind cavity 35.1 is provided on the most distal part of the lateral aspect of the lateral femoral condyle 34 (see FIG. 3) which means it is as close as possible to the knee joint. Although this has not been illustrated, each of the pair of femoral condyles 34 of the endoprosthesis 11 may be provided with a blind cavity 35.1 and an associated implantable electronic device 12 removably received therein.

Post-operatively, acceleration and joint rotation data recorded by the accelerometers 13 is collected and stored in memory 15. During a visit to a medical practitioner, the recorded and stored data can be downloaded to the remote computing device 18, which may be in the form of a smartphone, PDA, watch, wearable device, tablet, laptop, or other computing device, via the wireless communication module 14. The remote computing device 18 is configured to process the recorded data using suitable algorithms and/or artificial intelligence or machine learning techniques and to display the processed information to the medical practitioner. This may include information of activity patterns, potential issues associated with the prothesis i.e. looseness or instability, relative acceleration, relative rotation, relative tilt, vibration or force measured across the respective implantable electronic devices 12.1, 12.2. The medical prosthesis monitoring system 10 in accordance with the invention provides for more accurate monitoring of the endoprosthesis 11 and permits medical reporting of accurate, in situ, data that will contribute to the health of the patient.

The medical prosthesis monitoring system 10 gives a healthcare practitioner an unprecedented level of objective information on physical activity of the limb or replacement body part and gives practitioners the ability to gain easy access to accurate information specific to activity levels of the replacement body part or prosthesis. Furthermore, the activity information analysed and stored can possibly allow for earlier diagnosis of implant specific problems such as loosening of the prosthesis and activity patterns which may lead to a decline in general health of the patient. Other indirect benefits would include motivation of patients with accelerometer-enhanced implants to be more active.

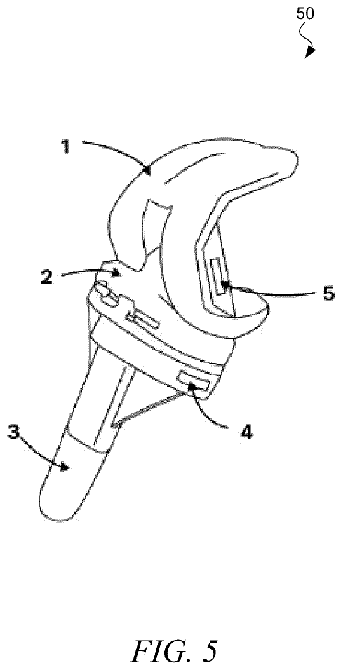

Another embodiment of an endoprothesis is designated by reference numeral 50 in FIG. 5. This endoprothesis 50 includes a femoral component 1, a tibial component 3 and an intervening lining or insert 2. Furthermore, an augment having a cavity 4 for receiving an implantable electronic device 12 therein has been attached to a lateral aspect of the tibial component 3. Likewise, another augment defining a cavity 5 has been attached to the lateral aspect of the femoral component 1. IMUs 13 may therefore be accommodated in cavities provided in augments attached to the respective components of the prosthesis 50.

The medical prosthesis monitoring system 10 provides for accurate long-term quantification of physical activity in patients following total knee replacement surgery. Using the memory 15 of the implantable electronic devices 12 activity can be recorded over a period of months or longer. Furthermore, permanent or semi-permanent introduction of the accelerometers 13 into the lateral aspects of the endoprosthesis 11 means that the accelerometers 13 can be disposed as close as possible to the knee without causing any discomfort to the user, which consequently results in more accurate measurements. Relative acceleration, rotation, and/or tilt may also be measured by having regard to measurement of the respective implantable electronic devices 12.1, 12.2.

As can best be seen in FIGS. 1 and 3, each implantable electronic device 12 is self-contained and comprises a one-piece, integrated, compact form factor. To this end, each implantable electronic device 12 has a casing 26 which defines an inner cavity for housing components of the implantable electronic device 12. Accordingly, the casing 26 is configured to house the accelerometer 13, memory 15, wireless communication module 14 and battery 16, amongst others, in the inner cavity. Accordingly, each implantable electronic device 12.1, 12.2 is self-contained and, as a whole, is configured to be removably mounted to the respective blind cavities 35.1, 35.2 of the knee prosthesis. The blind cavities 35.1, 35.2 are disposed in areas of the knee which has the least soft tissue cover which renders the implantable electronic devices 12.1, 12.2 easily removable, serviceable and/or replaceable by way of only minor surgery, without unnecessarily compromising structural integrity of the knee prosthesis itself.

The casing 26 includes two complementary, interconnectable parts 26.1, 26.2 which are operatively disconnectably coupled together to form a serviceable pod. The casing 26 may be manufactured from cobalt chrome molybdenum alloy, titanium or titanium alloy or any other material that is compatible with the material properties of the knee prosthesis.

To retain the implantable electronic devices 12.1, 12.2 in their respective blind cavities 35.1, 35.2, each blind cavity 35.1, 35.2 has a complementary cover 28.1, 28.2 which is removably secured over an opening of the blind cavity using either fasteners such as screws 29 which screw into holes or clips (not shown) which retain the cover 28 in position.

The Applicant believes that the endoprosthesis 11 has an advantage over other existing activity trackers for prosthetics due to the fact that, despite limited space and the load bearing function of the lateral condyle, provision of the blind cavities 35.1, 35.2 in the lateral aspect of the femoral or tibial condyle, improves accessibility to the self-contained, implantable electronic devices 12 and hence serviceability of these devices.

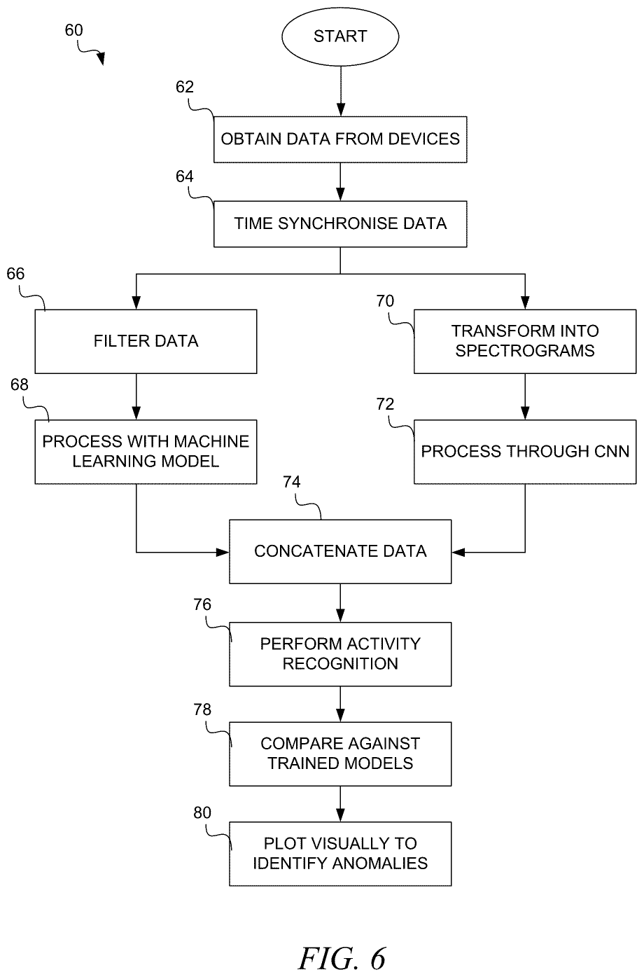

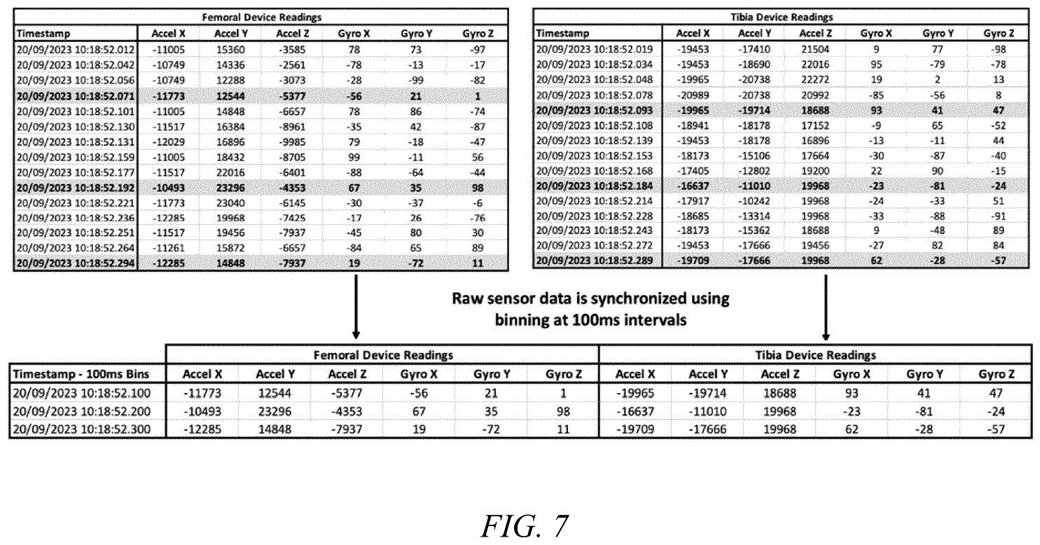

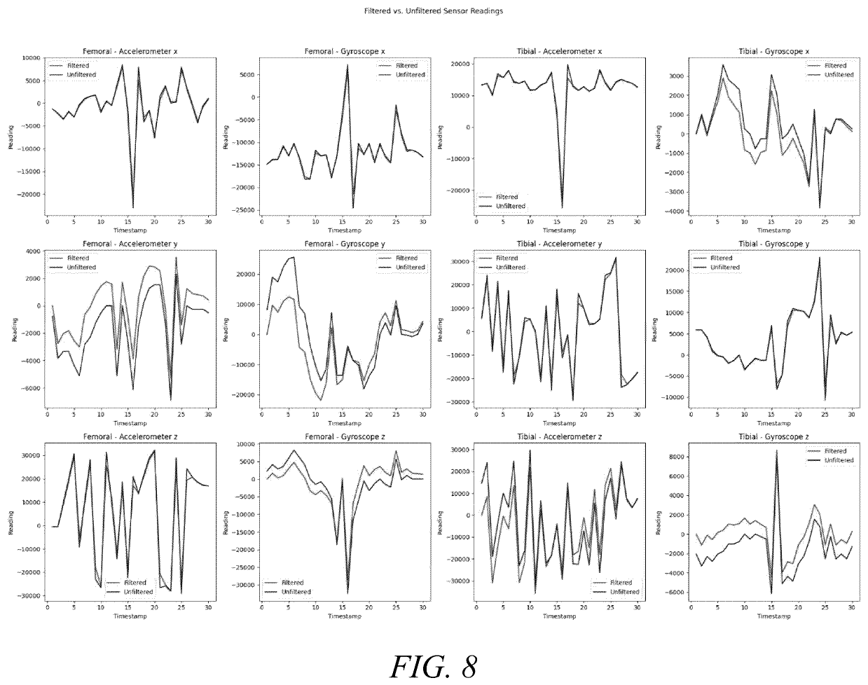

Reference is now made to FIG. 6 where reference numeral 60 refers generally to a method of monitoring a medical prosthesis 11 in accordance with the invention. As a first step in the monitoring process, data is obtained 62 from the respective implantable electronic device 12 using the remote computing device 18. Time synchonisation of the data received from the two devices 12 is performed at block 64. As can be seen in FIG. 7, the time-stamped data from the respective devices 12 is synchonised using binning at 100 ms intervals. The method 60 then involves performing temporal analysis of the data in a temporal branch shown on the left of FIG. 6, in parallel to performing temporal-spatial analysis of the data in a temporal-spatial branch shown on the right in FIG. 6. In the temporal branch the data undergoes a prefiltration step 66. Two kinds of data pre-processing is applied. First, a low-pass-filter is applied to the accelerometer raw data to remove high frequency noise and to modulate the effects of sudden movements and vibration. A high pass filter is then applied to remove drift and noise from the raw gyroscope data. FIG. 8 illustrates time-domain plots of filtered (red) and unfiltered (blue) data obtained from 12 channels of the devices 12.

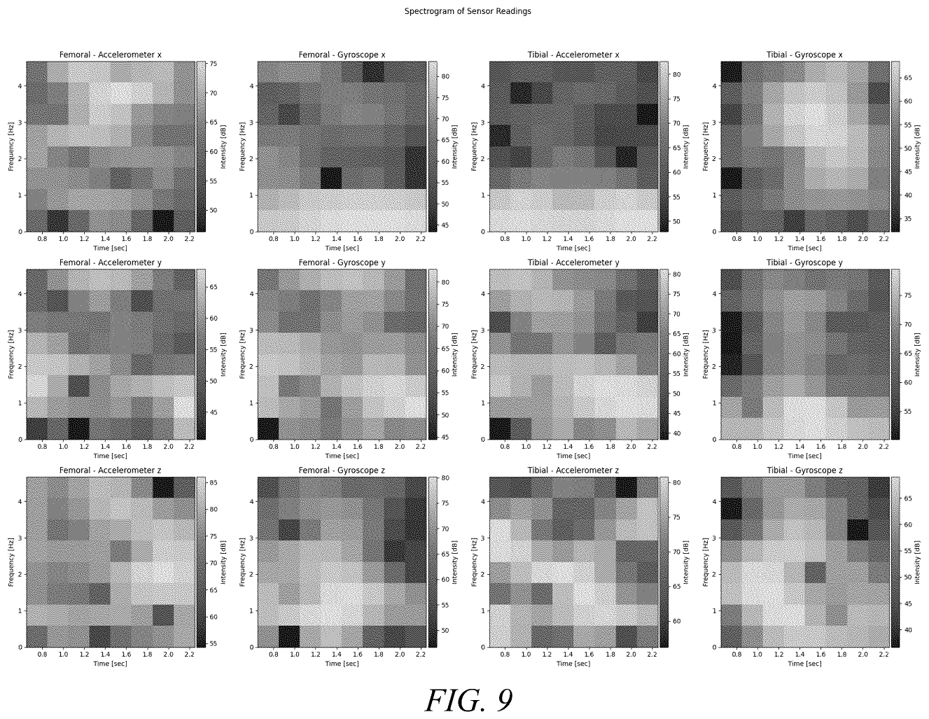

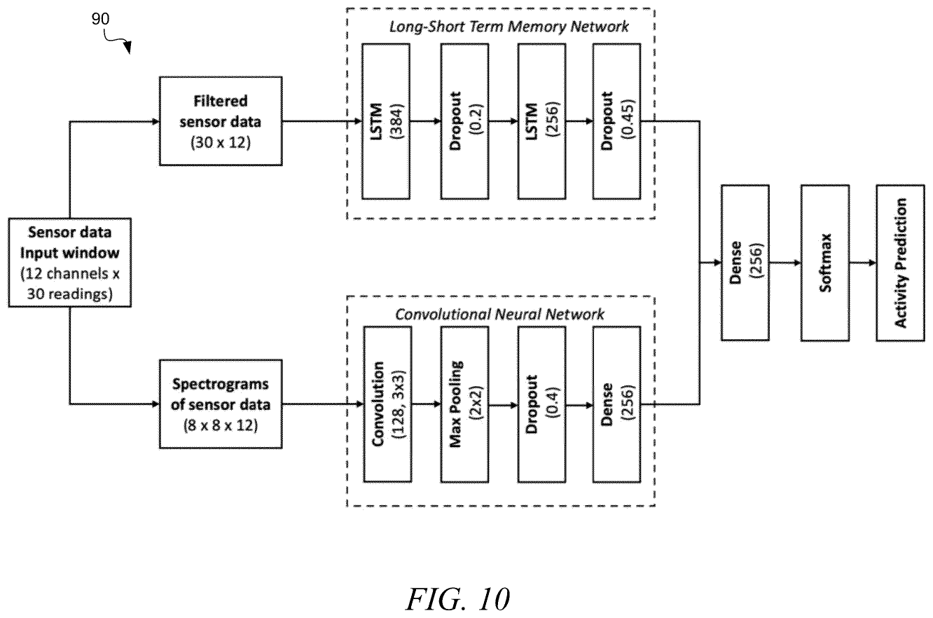

FIG. 10 shows a block diagram of machine learning model architecture 90 employed by the medical prosthesis monitoring system 10. As can been seen in FIG. 10, the model architecture 90 includes a Long-Short Term Memory (LSTM) machine learning network that processes, at block 68, filtered sensor data in the temporal branch. In combination with that the model architecture 90 includes, in the temporal-spatial branch, computing or transforming 70 the unfiltered data into spectrograms (see FIG. 9). As a next step in the method 60, the spectrograms are passed through a Convolutional Neural Network (CNN) at block 72. For the CNN, spectrograms are computed of each channel in each window to capture spatial patterns in frequency data using unfiltered data. Output data from the LSTM and CNN networks are concatenated 74 using late fusion to produce accurate activity predictions 76. The model architecture and its parameters were found using an extensive hyperparameter grid search. A multi-class classification algorithm (Softmax function) was used to create a probability distribution for all activity classes and a highest probability become the activity prediction.

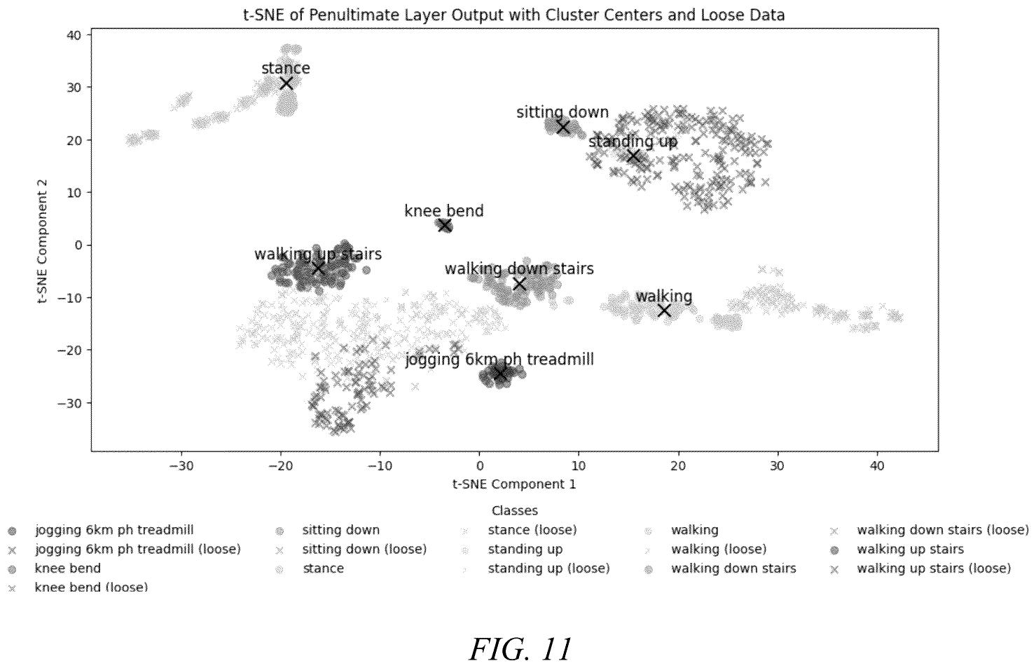

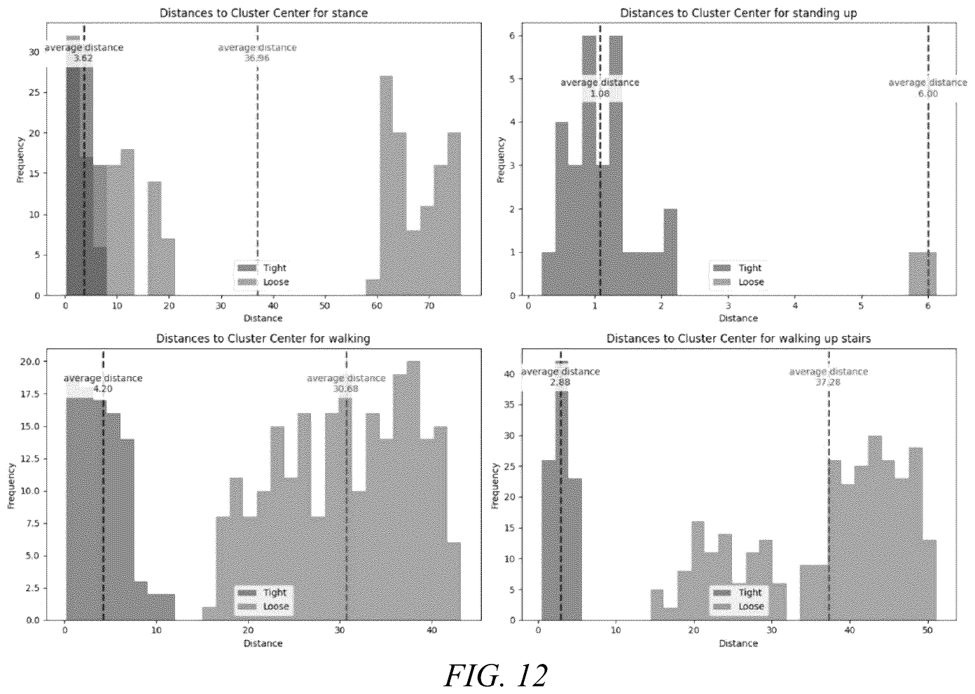

At block 78 recognised activities are compared against trained machine learning models in order to identify anomalies associated with the medical prosthesis 11. Finally, at block 80 output of the machine learning model 90 is plotted visually to help identify the anomalies. FIGS. 11 and 12 illustrate how classes of different activities are identified and grouped together and how a loose or malfunctioning prosthesis can be identified by having regarding the plotted output data.

The medical prosthesis monitoring system 10 and method 60 in accordance with the invention is capable of recognizing and predicting human knee activity based on movement data generated by dual IMU sensors in both the tibial and femoral components of a smart knee replacement prosthesis.

The method 60 includes synchronizing data, labelling activity, predicting activity and identifying prosthesis stability or other irregularities based on data generated by the dual IMUs 13 implanted in the knee replacement prosthesis.

Each IMU 13 consists of an accelerometer, gyroscope, magnetometer, and temperature sensor. Each IMU device is a six channel sensor that generates accelerometer and gyroscope data at, for example, 50 Hertz along the x, y and z axes. Understandably, a sampling frequency may vary in accordance with requirements. The implantable electronic devices are powered by a long-life battery power source and in some iterations has the capability to generate power from kinetic energy and can also be charged wirelessly through inductive coil technology. It has a Bluetooth transceiver, antenna and baseband processor and relays data wirelessly through low energy Bluetooth or other low energy intensive radiofrequency protocols within the acceptable medical spectrum. Each IMU device is placed within a blind chamber on the lateral aspect of the femoral and tibial components of a knee replacement prosthesis.

In some iteration, the IMU may be placed in a blind chamber in a step augment that is securely attached on to the lateral aspect of the femoral or tibial prosthesis. Step augments are commonly used to build up the knee prosthesis to substitute for bone loss during knee replacement surgery. The accelerometer and gyroscope data is streamed and analysed in real time by a Artificial Intelligence powered computer program to predict the activity of the knee. The system and method also has the capability of recognizing the activity based on analysis of stored IMU data.

IMU data is time-stamped for each IMU device. However, the generated data is not synchronized because of differences in the activation time of the IMUs.

To solve the problem of the disjointed datasets first, a simple algorithm is used to identify areas of overlap of the datasets where their time stamps overlap.

Then the data is binned into 100 ms bins to align the readings from the devices and generate sample windows of 30 readings. This defines a window size for sampling the two IMU datasets to create a single combined dataset. All 12 channels of the IMUs output is used to take advantage of all datapoints and thereby increase the level of accuracy of the system. This process is applied as data is streamed to allow instantaneous prediction of activity.

Activity pattern recognition based on machine learning using filtered timeseries data together with spectrograms of this data is a superior solution and allows for effective use of established machine learning techniques. The model 90 learns to capture temporal dependencies through its LSTM branch and its CNN captures spatial patterns in the transformed spectrogram representations of the sensor data. By employing a late fusion approach, the model effectively integrates the features learned by each branch to accurately recognize activities without needing to manually adjust thresholds.

A combination of programmatic labelling and training data from direct simulation of a smart knee replacement prosthesis, cadaveric smart knee replacement simulation, healthy patients and patients with well-functioning total knee replacements executing the defined activities of daily living was used to train the model.

A combination of semi-supervised and active learning for the programmatic labelling was used.

### Direct Simulation of a Smart Knee Device

A smart knee replacement device with dual IMU devices in chambers on the lateral aspect of the femoral and tibial components of the prosthesis was attached to a knee simulator (AMTI VIVO, Boston Massachusetts) and the simulator was used to execute specified ADL. The VIVO simulator is a hydraulic device with 360 degree range freedom of movement that has been designed for human joint movement simulation. Data from an open source library, Orthoload (Orthoload, Germany) was used for the typical forces acting on a knee to input appropriate loads for the smart knee prosthesis throughout the simulated ADL. Data was collected from a minimum of 100 cycles per specified ADL for analysis.

Simulation of a Smart Knee Device Implanted into a Cadaver

A cemented smart knee replacement device was implanted into a cadaver knee. The cadaver knee was attached to the VIVO simulator and run through 100 cycles per specified ADL under force loading conditions imported from Orthoload. The data was subsequently collected for analysis.

### Healthy Subjects

The IMU devices in purpose-built housings were attached with straps to the lateral aspect of the distal femur and proximal tibia of healthy individuals without any knee complaints or prior history of lower limb injury or surgical intervention to the limb. The IMU devices that are attached to the skin are subject to the vagaries of soft tissue movement. This can introduce “noise” to the output data and highlights the need for data pre-processing to reflect true skeletal movement. The subjects underwent a minimum of 100 cycles per specified ADL.

### Well-Functioning Knee Replacements

Subjects with well-functioning posterior stabilized cemented knee replacements were identified and recruited. The IMU devices were strapped as in the healthy subjects and data generated was collected for analysis. The subjects underwent a minimum of 100 cycles per specified ADL.

LSTM networks are a subset of recurrent neural networks. They leverage past values to predict future trends. Traditional recurrent networks are limited by the vanishing and exploding gradient phenomena. They are not efficient at learning and retaining information over long sequences and consequently have impaired ability to retain long-term information.

With reference to FIG. 10, a dropout function was applied after each LSTM layer to prevent overfitting. The dropout layers were applied at a rate of 0.2 and 0.45 respectively. During the training phase the first LSTM's output has a 20% probability of being set to zero randomly at each update and similarly 45% probability for the second LSTM layer. The dropout is applied to the output feature map of the respective LSTM across all timesteps, effectively reducing the number of features available to the next layer at each timestamp. This mitigates reliance on any single input node, promoting the learning of robust features that generalize well. The LSTM was trained using standard backpropagation through time (BPTT). Max pooling with a 2×2 matrix and a dropout function of 0.4 was used in the CNN. A progressively higher accuracy of activity prediction was found using the LSTM model alone compared to the CNN alone and finally the fusion matrix.

Stability of the implants refers to fixation of implants to underlying bone. Typically, fixation is achieved with use of polymethylmethacrylate (PMMA) bone cement in the interface between the prosthesis and the patient's bone at the time of surgical implantation of the prosthesis or use of cementless implants with a porous inner layer that allows bone ingrowth and ongrowth and secure anchorage in the short term. A common mode of failure of knee replacements is loss of fixation over time. This fate can affect either or both the femoral and tibial prosthesis. The machine learning model 90 that has been developed in this invention achieves 98% out-of-sample test accuracy for activity recognition in stable knee prosthesis. The model combines LSTM network running on filtered input data windows with a CNN operating on spectrograms of the unfiltered input data. These two networks are combined using late fusion with dense layers and a softmax. The dense layers are then used as embeddings in a few-shot learning model. The few-shot machine leaning framework was used to make accurate predictions based on a small number of labelled activity samples.

Embeddings were collected from the activity recognition model based on stable implants. A K-means clustering machine learning algorithm was used to partition the datasets. For each activity, the datasets were initialized by determining the centroids formed by the clusters representing activity patterns per activity class. Euclidean distance was then used for assignment to form the clusters representing activity patterns per activity class. New centroids were calculated by taking a mean of all data points assigned to each cluster and iteratively updating the centroids until they did not change significantly.

Then the embedding vector for sample windows from unstable implants was computed and distances between them were measured and centroids were established. This correctly identifies the activity and importantly, allows the inference to be made of instability of the prosthesis based on the distance of the embedded point from the cluster center as shown in the T-SNE plot in FIG. 11. Distance between the input sample's embedding and the nearest activity specific cluster centroid is a surrogate for the magnitude of instability of the prosthesis.

Ideally, baseline embeddings should be calculated for activity data of a new patient at the time when the prosthesis would be expected to be stable and activity levels have returned to normal-around three to six months for most patients. The embeddings would then be fed into the trained model and would allow establishment of thresholds and definition of envelope of normality to increase the sensitivity of detection of instability of the implants.

By analysing distance between the embeddings of patient activity data and centroids representing stable knee replacement behaviour, the system 10 can recognize instability in either or both the femoral and tibial prosthesis of a knee replacement. Furthermore, inference can be made of the degree of such instability.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and/or computer program products according to embodiments of the invention.

It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create modules for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process (or method), such that the instructions which execute on the computer, other programmable apparatus, or other device implement the functions/acts specified in the flowchart and/or block diagram block or blocks.

The flowchart and/or block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The use of dual IMUs (femoral and tibial) with a synchronization pipeline to align data streams achieves 96% accuracy vs ˜76-82% for single sensors, an ˜80% reduction in activity prediction error compared to single-sensor approaches.

This quantifiable improvement is a strong technical differentiator of the system 10 and method 60 in accordance with the invention. Furthermore, the Applicant believes that the multimodal machine learning architecture combining an LSTM branch on filtered time-series with a CNN on spectrograms, fused at a late integration stage is novel over the prior art. Furthermore, the system 10 addresses a unique problem of disjoined datasets across sensors by introducing a temporal synchronisation algorithm for aligning two independent IMU data sets. This algorithm includes binning, windowing, and dual pre-processing streams. Contrary to what the prior art teaches, the system 10 and method 60 in accordance with the invention leverages the activity recognition model to embed observation windows, then applies cluster-distance anomaly detection to flag loosening/instability of the prosthesis. The Applicant believes that this embedding-based anomaly detection approach adds predictive diagnostic capability which goes beyond what the prior art can do.

1. A computer-implemented method of monitoring a medical prosthesis which includes at least two implantable electronic devices implanted in the medical prothesis, the method including:
obtaining data from the implantable electronic devices;
performing, using a computing device, temporal-spatial analysis of the obtained data;
performing activity recognition using machine learning techniques; and
comparing recognised activities against trained machine learning models in order to identify anomalies associated with the medical prosthesis.

2. The computer-implemented method as claimed in claim 1, which includes the prior step of synchronizing time-stamped data obtained from both of the respective implantable electronic devices.

3. The computer-implemented method as claimed in claim 1, wherein the step of performing temporal-spatial analysis of the obtained data includes transforming, using the computing device, the obtained data into spectrograms.

4. The computer-implemented method as claimed in claim 3, which includes processing, using the computing device, the spectrograms through a convolutional neural network in order to identify specific temporal-spatial patterns.

5. The computer-implemented method as claimed in claim 1, wherein the step of performing temporal-spatial analysis includes:
performing temporal analysis of the obtained data using a machine learning model in a temporal branch; and,
in parallel to that, performing temporal-spatial analysis of the obtained data using spectrograms in a temporal-spatial branch.

6. The computer-implemented method as claimed in claim 5,
wherein performing temporal analysis in the temporal branch includes the prior step of filtering the obtained data; and
wherein performing temporal-spatial analysis in the temporal-spatial branch includes processing the spectrograms through a trained convolutional neural network in order to identify specific temporal-spatial patterns.

7. The computer-implemented method as claimed in claim 5, which includes late-fusing the respective branches by concatenating processed data from the temporal branch and the temporal-spatial branch to integrate features learned by each branch in order to accurately recognize activity patterns without needing manually to adjust thresholds.

8. The computer-implemented method as claimed in claim 7, wherein performing activity recognition includes creating a probability distribution for all activity classes, using a multi-class classification algorithm, such that a highest probability in the distribution becomes the activity prediction.

9. The computer-implemented method as claimed in claim 7, wherein the step of comparing recognised activities against trained machine learning models includes using a K-means clustering machine learning algorithm to partition datasets.

10. The computer-implemented method as claimed in claim 9, which includes using t-Distributed Stochastic Neighbor Embedding (t-SNE) plotting visually to identify anomalies associated with the medical prosthesis.

11. The computer-implemented method as claimed in claim 7, which includes performing cluster-distance anomaly detection by plotting distances to cluster centres for recognised activities.

12. The computer-implemented method as claimed in claim 1, wherein the medical prosthesis is a knee prosthesis.

13. The computer-implemented method as claimed in claim 1, which includes the prior step of training the machine learning models using data obtained from patients with well-functioning medical protheses.

14. A medical prosthesis monitoring system which includes:
at least two implantable electronic devices mounted to a medical prothesis fitted to, or mountable to a subject; and
a remote computing device which is communicatively coupled to a wireless communication module of the implantable electronic devices and is configured to:
wirelessly interrogate the implantable electronic devices to obtain data measured by electromechanical motion sensors of the implantable electronic devices;
perform temporal-spatial analysis of the obtained data;
perform activity recognition using machine learning techniques; and
compare recognised activities against trained machine learning models in order to identify anomalies associated with the medical prosthesis.

15. The medical prosthesis monitoring system as claimed in claim 14, wherein at least one of the implantable electronic devices is implanted into an augment attached to part of the medical prosthesis.

16. The medical prosthesis monitoring system as claimed in claim 14, wherein the remote computing device is further configured to transform, using a processor, the obtained data into spectrograms.

17. The medical prosthesis monitoring system as claimed in claim 16, wherein the remote computing device is further configured to:
perform, using the processor, temporal analysis of the obtained data using a machine learning model in a temporal branch; and,
in parallel to that, perform, using the processor, temporal-spatial analysis of the obtained data using spectrograms in a temporal-spatial branch.

18. The medical prosthesis monitoring system as claimed in claim 17, wherein the remote computing device is further configured to perform cluster-distance anomaly detection by plotting distances to cluster centres for recognised activities.

19. A non-transitory computer-readable storage medium having program instructions stored thereon, which, when executed by a computing device, enable the computing device to perform the steps of the computer-implemented method of claim 1.
